(19) United States
(12) Patent Application Publication
(10) Pub. No.: US 2021/0137713 A1
(43) Pub. Date: May 13, 2021

(54) STENT DELIVERY DEVICE

(71) Applicants: Kyong Min SHIN, Gyeonggi-do (KR); TAEWOONG MEDICAL CO., LTD, Gyeonggi-do (KR)

(72) Inventors: Kyong Min SHIN, Gyeonggi-do (KR); Dong Ho SHIN, Seoul (KR)

(21) Appl. No.: 17/044,456

(22) PCT Filed: Apr. 25, 2019
(86) PCT No.: PCT/KR2019/004975

§ 371 Date: Oct. 1, 2020

(30) Foreign Application Priority Data
May 10, 2018 (KR) 10-2018-0053850

(51) Int. Cl. A61F 2/95; A61F 2/966

(52) U.S. Cl. CPC A61F 2/9517; A61M 25/09; A61F 2/966

(57) ABSTRACT

A stent delivery device includes: a knob having a first passage a fitting portion; a handle rotatably installed on the fitting portion and having a winding portion; a guide member having a second passage, a fitting hole and an insertion, a first guide hole and a second guide hole; an inner tube inserted into the first and second passages and exposed, a guide tip being installed on one side of the inner tube so as to form a mounting space in which the stent is mounted; an outer tube movably installed on the outer surface of the inner tube, a connecting tip being provided on the outer surface of the outer tube; and a pulling wire connected to the winding portion and to the connecting tip of the outer tube through the first and second guide holes.

This application is a national stage application of PCT/KR2019/004975 filed on Apr. 25, 2019, which claims priorities of Korean patent application number 10-2018-0053850 filed on May 10, 2018. The disclosure of each of the foregoing applications is incorporated herein by reference in its entirety.

### TECHNICAL FIELD

The present invention relates to a stent delivery device, and more particularly, to a stent delivery device for positioning and installing a stent at a constricted or closed lesion part during an operation more conveniently and stably than when using conventional devices.

### BACKGROUND ART

In general, when a constricted or closed lesion part occurs in a lumen in the body, such as the esophagus, the duodenum, the biliary tract, the urethra or the ureter, its own intrinsic function of moving a body fluid is lowered.

Therefore, a stent is conventionally inserted into the position of the constructed or closed lesion part through a stent delivery device, thereby expanding the narrowed lumen.

In this regard, Patent Document 1 provides an artificial vessel stent insertion device for a vascular surgical operation which inserts a moving tube provided with one side end, to which a knob is fixed, into an outer tube in front of a connector so as to be moved forwards and backwards, connects a connection tube provided with an insertion end formed at one end thereof to the front end of the moving tube, and inserts a hollow-type cylindrical artificial blood vessel stent, adjusted to have minimum diameter, between the connection tube and the outer tube so as to insert the artificial blood vessel stent into a lesion part of a blood vessel, and an artificial vessel stent insertion device is characterized in that a wire hole is formed through the front end of the connection tube so that one side of each of a plurality of wires is inserted into the wire hole in a a loop form, an iron core extending from the inside of the connection tube and protruding to the outside of the knob is inserted into the wire loop, and the other side of each of the wires is fixed so as to surround the front end of the artificial blood vessel stent.

However, in Patent Document 1, the connector connected to the outer tube must be directly pulled by external force in order to install the stent at the constricted or closed lesion part and, if a surgeon is unfamiliar with this operation, the connector may be unstably pulled.

That is, there is a risk of not properly placing and installing the stent at the lesion part. In other words, there may be a risk of failure of the operation.

### RELATED ART DOCUMENT

### Patent Document

Patent Document 1: Korean Utility Model Registration No. 20-0312490 (Registration Date: Apr. 23, 2003)

### DISCLOSURE

### Technical Problem

Therefore, the present invention has been made in view of the above problems, and it is an object of the present invention to provide a stent delivery device for positioning and installing a stent in a constricted or closed lesion part during an operation more conveniently and stably than when using conventional devices.

### Technical Solution

In accordance with the present invention, the above and other objects can be accomplished by the provision of a stent delivery device including a knob configured to have a first passage formed therein and a fitting portion formed to protrude therefrom, a handle rotatably installed on the fitting portion and configured to have a winding portion formed to protrude therefrom, a guide member configured to have a second passage formed therein and connected to the first passage, a fitting hole formed on a periphery of the second passage such that the fitting portion is fitted into the fitting hole, an insertion hole formed on the periphery of the second passage such that the winding portion inserted into the insertion hole, a first guide hole formed through one side of an outer surface of the guide member, and a second guide hole formed through a remaining side of the outer surface of the guide member so as to be connected to the insertion hole, an inner tube inserted into the first and second passages and then exposed therefrom, and configured to have a guide tip installed on one side thereof so as to form a mounting space in which a stent is mounted, and outer tube movably installed on the outer surface of the inner tube and configured to have a connecting tip provided on the outer surface thereof, and a pulling wire connected the winding portion and the connecting tip of the outer tube through the first and second guide holes.

### Advantageous Effects

A first embodiment of the present invention is effective in installing a stent in a lesion part by pulling an outer tube using a pulling wire through a handle which rotated about an inner tube.

That is, the outer tube may be pulled simply by rotating the handle regardless of familiarity of hands and strength of pulling force of hands, and thus, the stent may be positioned and installed in the lesion part more conveniently and stably than when using conventional devices.

The first embodiment of the present invention prevents twisting of the pulling wire caused by rotation of the handle through first and second guide holes of a guide member, and is thus effective in preventing the outer tube from not moving or from rotating about the inner tube when the handle is rotated.

That is, expansion of the stent installed in the lesion part is not impeded.

The first embodiment of the present invention prevents exposure of the pulling wire to the outside through a guide tube, and is thus effective in preventing the pulling wire from interfering with other medical devices during an operation.

The embodiment of the present invention guides a knob wing to a third guide hole, and is thus effective in further preventing the outer tube from being rotated due to pulling of the pulling wire during the operation.

Further, after the operation is completed, mounting of a new stent the mounting space of the inner tube is facilitated.

In the first embodiment of the present invention, an insertion protrusion of a guide member is inserted into an insertion groove of a knob, and thus, rotation of the knob and the guide member due to friction caused by rotation of the handle during the operation is prevented.

The first embodiment of the present invention prevents user's fingers from sliding on the handle during the operation through knurled parts, and is thus effective in appropriately performing the operation.

The first embodiment of the present invention is effective preventing a guide wire from being caught on the inner tube through extension portion when the guide wire is inserted into the inner tube.

Further, regardless of the direction in which the guide wire is inserted into the extension portion, the guide wire is guided to be directly inserted into the inner tube by a guide plane.

In first embodiment of the present invention, the handle may be automatically rotated using a driving member, and thus, a user may more simply and precisely perform the operation.

That is, the stent may be positioned and installed in the lesion part more conveniently and stably than when using the conventional devices.

Further, the handle may be manually rotated through a manual switching button.

That is, in response to a defect the driving member or depending on the operation situation, the user may select one of automatic rotation and manual rotation of the handle.

The first embodiment of the present invention, when an iron core is inserted into the inner tube, preemptively prevents the inner and outer tubes from being bent by external force applied from the outside prior to the operation or from being damaged due to excessive bending thereof.

That is, safety management of the inner and outer tubes is strengthened.

Further, the iron core may be easily installed in or released from the inner tube through knob head.

A second embodiment of the present invention is effective in installing a stent in a lesion part by pulling an outer tube using a pulling wire through a handle which is not rotated about an inner tube, i.e., which is rotated unlike the first embodiment.

That is, the outer tube may be pulled simply by rotating the handle regardless of familiarity of hands and strength of pulling force of hands, and thus, the stent may be positioned and installed in the lesion part more conveniently and stably than when using conventional devices.

The second embodiment of the present invention prevents twisting of the pulling wire caused by rotation of the handle through a first guide hole of a knob and a second guide hole a guide member, and is thus effective in preventing the outer tube from not moving or from rotating about the inner tube when the handle is rotated.

That expansion of the stent installed in the lesion part is not impeded.

The second embodiment of the present invention prevents exposure of the pulling wire to the outside through the guide member, and is thus effective in preventing the pulling wire from interfering with other medical devices during an operation.

The second embodiment of the present invention guides a knob wing to a third guide hole, and is thus effective in preventing the outer tube from being rotated due to pulling of the pulling wire during the operation.

Further, after the operation is completed, mounting of a new stent in the mounting space of the inner tube is facilitated.

The second embodiment of the present invention prevents user's fingers from sliding on the handle during the operation through knurled parts, and is thus effective in appropriately performing the operation.

The second embodiment of the present invention is effective in preventing a guide wire from being caught on the inner tube through an extension portion when the guide wire is inserted into the inner tube.

Further, regardless the direction in which the guide wire is inserted into the extension portion, the guide wire is guided to be directly inserted into the inner tube by a guide plane.

The second embodiment of the present invention may automatically rotate the handle using a driving member, and thus a user may more simply and precisely perform the operation.

That is, the stent may be positioned and installed in the lesion part more conveniently and stably than when using the conventional devices.

Further, the handle may be manually rotated through a manual switching button.

That is, in response to a defect in the driving member or depending on the operation situation, the user may select one of automatic rotation and manual rotation of the handle.

The second embodiment of the present invention, when as iron core is inserted into the inner tube, preemptively prevents the inner and outer tubes from being bent by external force applied from the outside prior to the operation or from being damaged due to excessive bending thereof.

That is, safety management the inner and outer tubes is strengthened.

Further, the iron core may be easily installed in or released from the inner tube through a knob head.

### DESCRIPTION OF DRAWINGS

FIGS. 1 to 3 are perspective views of a stent delivery device according to a first embodiments of the present invention.

FIGS. 4A, 4B, 5A, 5B and 5C are views illustrating the stent delivery device according to the first embodiment of the present invention in more detail.

FIGS. 6A and 6B views illustrating a stent delivery device according to a modified embodiment of the first embodiment of the present invention in more detail.

FIGS. 7A to 10 are views illustrating the usage state of the stent delivery device according to the first embodiment of the present invention.

FIG. 11 is a perspective view of a stent delivery device according to a second embodiment of the present invention.

FIGS. 12A, 12B, 13A, 13B, and 13C are views illustrating the stent delivery device according to the second embodiment of the present invention in more detail.

FIGS. 14A and 14B views illustrating a stent delivery device according to a modified embodiment of the second embodiment of the present invention in more detail.

FIGS. 15A to 18 are views illustrating the usage state of the stent delivery device according to the second embodiment of the present invention.

### BEST MODE

Hereinafter, reference will now be made detail to embodiments of the present invention, examples of which are illustrated in the accompanying drawings.

As shown in FIGS. 1 to 18, stent delivery devices 100 and 200 according to first and second embodiments of the present invention are used to install a stent 1 at a constricted or closed lesion part occurring in a lumen in the body, such as the esophagus, the duodenum, the biliary tract, the urethra or the ureter, so as to expand the constricted or closed lesion part.

Further, as shown in FIGS. 1 to 10, the stent delivery device 100 according to the first embodiment of the present invention includes a knob 110, which has a first passage 111 formed therein and a fitting portion 112 formed to protrude therefrom, and a handle 120, which is rotatably installed on the fitting portion 112 and has a winding portion 121 formed to protrude therefrom.

Here, the handle 120 has knurled parts 122, which are a plurality of recesses formed in the outer surface thereof so as to be disposed at predetermined intervals, and is rotated about the fitting portion 112.

Further, at least one insertion groove 112a is formed in the outer surface of the fitting portion 112.

In addition, the stent delivery device 100 includes a guide member 130, which has a second passage 131 formed therein and connected to the first passage 111, a fitting hole 132 formed on the periphery of the second passage 131 such that the fitting portion 112 is fitted into the fitting hole 132, an insertion hole 133 formed on the periphery of the second passage 131 such that the winding portion 121 is inserted into the insertion hole 133, at least one first guide hole 134 formed through one side of the outer surface of the guide member 130, and at least one second guide hole 135 formed through the opposite side of the outer surface of the guide member 130 so as to be connected to the insertion hole 133.

Here, at least one insertion protrusion 132a, which is inserted into the insertion groove 112a, protrudes from the inner surface of the fitting hole 132.

Further, the first guide holes 134 formed through one side of the outer surface of the guide member 130 so as to be inclined in a direction towards the center of the guide member 130, i.e., the second passage 131, and the second guide holes 135 are formed through the opposite side of the outer surface of the guide member 130 in a direction towards the portion of the guide member 130 that is orthogonal to the second passage 131, i.e., the winding portion 121.

Further, a guide tube 136, which has a third passage 136a formed therein and connected to the second passage 131 and the first guide holes 134, protrudes from the guide member 130.

Moreover, at least one third guide hole 136b is formed through the outer surface of the guide tube 136.

In addition, the stent delivery device 100 includes an inner tube 140 which is sequentially inserted into the first, second and third passages 111, 131 and 136b, is exposed therefrom, and has a guide tip 141 installed on one side of the inner tube 140 so as to form a mounting space 142 in which the stent 1 is mounted.

Here, an extension portion 143 having a diameter greater than the diameter of the inner tube 140 protrudes from the other side of the inner tube 140 opposite the guide tip 141.

Further, a guide plane 143a is formed on the inner surface of the extension portion 143 so as to be inclined in a direction towards the inner tube 140.

Further, the guide tip 141 has a passage formed therein and connected to the inner tube 140, and has a diameter greater than the diameter of the inner tube 140.

In addition, the stent delivery device 100 includes an outer tube 150, which is movably installed on the outer surface of the inner tube 140, has a connecting tip 150a provided on the outer surface of the outer tube 150, and is caught on the guide tip 141.

Here, at least one knob wing 151 guided along the third guide hole 136b is installed around the connection tip 150a of the outer tube 150.

Further, the outer tube 150 compresses the stent 1 mounted in the mounting space 142.

Moreover, the stent delivery device 100 includes at least one pulling wire 160, which is connected to the winding portion 121 and the connecting tip 150a of the outer tube 150 through the first and second guide holes 134 and 135.

Here, the pulling wire 160 is inserted into the third passage 136a.

Further, the stent delivery device 100 includes a guide wire 170 which is inserted into the inner tube 140 and the guide tip 141 and is then exposed therefrom.

Here, the guide wire 170 is guided by the guide plane 143a through the extension portion 143, is inserted into the inner tube 140, and is then exposed from the guide tip 141, when an operation is performed.

Further, the stent delivery device 100 includes an iron core 190 which is inserted into the inner tube 140 through the guide tip 141.

Here, the iron core 190 has a knob head 191 which is exposed from the guide tip 141, and maintains an inserted state thereof into the inner tube 140 before the operation is performed.

Further, as shown in FIG. 6, a stent delivery device 100 according to a modified embodiment of the first embodiment of the present invention includes a driving member 180 which is installed in a knob 110 so as to rotate a handle 120 in forward and reverse directions.

Here, the driving member 180 includes a servomotor, a stepping motor or the like which transmits power to rotate the handle 120 in the forward and reverse directions, and includes a button configured to control the motor.

Further, the stent delivery device 100 includes a manual switching button 181 which is installed on the knob 110, and stops driving of the driving member 180 so as to manually rotate the handle 120.

That is, when the manual switching button 181 is touched once, the driving member 180 is in a drivable state, and when the manual switching button 181 is touched twice, the driving member 180 is in an undrivable state.

Further, when the servomotor or the stepping motor included in the driving member 180 is defective, it may be removed or replaced through an outlet formed in the knob 110.

As shown FIGS. 11 to 18, the stent delivery device 200 according to a second embodiment of the present invention includes a knob 210, which has a first passage 211 formed therein and an installation hole 212 and at least one first guide hole 213 connected to the installation hole 212, the installation hole 212 and the at least one first guide hole 213 being formed in the outer surface thereof, and a handle 220, which has a winding portion 221 rotatably installed in the installation hole 212 so as to protrude therefrom and exposed from the installation hole 212.

Here, the knob 210 has are similar to a box, and the first guide hole 213 is formed in the outer surface of the knob 210 in a curved shape in a direction towards the winding portion 221.

Further, the handle 220 has knurled parts 222, which are a plurality of recesses formed in the outer surface thereof so as to be disposed at predetermined intervals.

In addition, the stent delivery device 200 includes an inner tube 230 which is inserted into the first passage 211, is exposed therefrom and has a guide tip 231 installed on one side of the inner tube 230 so as to form a mounting space 232 in which a stent 1 is mounted.

Here, an extension portion 233 having a diameter greater than the diameter of the inner tube 230 protrudes from the other side the inner tube 230 opposite the guide tip 231.

Further, a guide plane 233a is formed on the inner surface of the extension portion 233 so as to be inclined in a direction towards the inner tube 230.

Further, the guide tip 231 has a passage formed therein and connected to the inner tube 230, and has a diameter greater than the diameter of the inner tube 230.

In addition, the stent delivery device 200 includes an outer tube 240, which is movably installed on the outer surface of the inner tube 230, has a connecting tip 240a provided on the outer surface of the outer tube 240, and is caught on the guide tip 231.

Here, at least one knob wing 241 installed around the connection tip 240a of the outer tube 240.

Further, the outer tube 240 compresses the stent 1 mounted in the mounting space 232.

Moreover, the stent delivery device 200 includes at least one pulling wire 250, which is connected to the winding portion 221 and the connecting tip 240a of the outer tube 240 through the first guide hole 213.

Further, the stent delivery device 200 includes a guide wire 260 which is inserted into the inner tube 230 and the guide tip 231 and is then exposed therefrom.

Here, the guide wire 260 is guided by the guide plane 233a through tale extension portion 233, is inserted into the inner tube 230, and is exposed from the guide tip 231, when an operation is performed.

Further, the stent delivery device 200 includes a guide member 270, which is installed outside the knob 210 and has a second passage 271 formed therein and connected to the first passage 211 and at least one second guide hole 272 formed in the outer surface thereof and connected to the first guide hole 213 and the second passage 271.

Here, the pulling wire 250 is inserted into the second passage 271 and the second guide hole 272, and the inner tube 230 inserted into the second passage 271.

Further, at least one third guide hole 273 configured to guide the knob wing 241 is formed through the outer surface of the guide member 270.

In addition, the stent delivery device 200 includes an iron core 290 which is inserted into the inner tube 230 through the guide tip 231.

Here, the iron core 290 has a knob head 291 which is exposed from the guide tip 231, and maintains an inserted state thereof into the inner tube 230 before the operation is performed.

Further, as shown FIG. 14, a stent delivery device 100 according to modified embodiment of the second embodiment of the present invention includes a driving member 280 which is installed in a knob 310 so as to rotate a handle 320 in forward and reverse directions.

Here, the driving member 280 includes a servomotor, a stepping motor or the like which transmits power to rotate the handle 220 in the forward and reverse sections, and includes a button configured to control the motor.

Further, the stent delivery device 200 includes a manual switching button 281 which is installed on the knob 210 and stops driving of the driving member 280 so as to manually rotate the handle 220.

That is, when the manual switching button 281 is touched once, the driving member 280 is in a drivable state, and when the manual switching button 281 is touched twice, the driving member 280 is in an undrivable state.

Further, when the servomotor or the stepping motor included in the driving member 280 is defective, it may be removed or replaced through an outlet formed in the knob 210.

The operation of the above-described stent delivery devices 100 and 200 according to the first and second embodiments of the present invention will be described below.

As shown FIGS. 1 to 10, in the stent delivery device 100 according to the first embodiment of the present invention, in order to perform an operation, the knob head 191 is gripped and thereby the iron core 190 is easily removed from the inner tube 140 and the guide 141.

When the iron core 190 is removed, the inner tube 140 and the outer tube 150 may be in a free state in which they are bendable corresponding to a lumen in the body.

Thereafter, the guide wire 170 is easily inserted into the inner tube 140 through the extension portion 143, and is exposed from the guide tip 141.

Here, the guide wire 170 is guided by the guide plane 143a of the extension portion 143, thereby being more easily inserted into the inner 140.

Thereafter, after a site to be operated on is confirmed by inserting an endoscope into an incision partially formed in the lumen of the body, the guide wire 170 is pushed into the site to be operated on, i.e., a lesion part.

Thereafter, the outer tube 150 is moved to the site to be operated on along the guide wire 170 by pushing the knob 110 and the knob wings 151.

Here, the guide tip 141 connected to the inner tube 140 is caught on the outer tube 150 moved by the knob wings 151, and is thus moved to the site to be operated on along the guide wire 170.

Thereafter, in the state in which a user grips the knob 110, the user rotates the handle 120 about the fitting portion 112 and the inner tube 140 and thus slowly pulls the pulling wires 160.

Here, the user's fingers rotating the handle 120 are prevented from slipping on the handle 120 by the knurled parts 122.

Then, the pulling wires 160 are wound on the winding portion 121, thus pulling the connection tip 150a of the outer tube 150.

Here, the outer tube 150 is slowly moved backwards by the handle 120 which slowly winds the pulling wires 160 on the winding portion 121.

Further, the pulling wires 160 are moved backwards along a straight path in the third passages 136a of the guide tube 136, without twisting due to the first and second guide holes 134 and 135 of the guide member 130.

Further, the knob wings 151 are guided to be moved backwards by the third guide holes 136b of the guide tube 136, and thus, the pulling wires 160 are moved backwards along a straighter path in the third passages 136a of the guide tube 136.

Of course, as shown in FIG. 6, the pulling wires 160 may be pulled by automatically rotating the handle 120 through driving of the driving member 180, without using the user's fingers.

Alternatively, after driving of the driving member 180 is stopped through the manual switching button 181, the pulling wires 160 may be pulled by manually rotating the handle 120.

Thereby, the stent 1 mounted in the mounting space 142 is expanded by backward movement of the outer tube 150 achieved by winding the pulling wires 160 on the winding portion 121.

That is, the stent 1 is located in the constricted or closed lesion part and is then expanded, by the stent delivery device 100 according to the first embodiment of the present invention.

Thereafter, when installation of the stent 1 is completed, the inner and outer tubes 140 and 150 and the guide wire 170 of the stent delivery device 100 are removed from the lumen in the body, and then a new stent 1 is mounted in the mounting space 142 of the inner tube 140.

Then, the guide wire 170 is pulled out from the inner tube 140, and the pulling wires 160 wound on the winding portion 121 are loosened by rotating the handle 120 in the reverse direction.

Thereafter, the knob wings 151 are gripped and then moved forwards along the third guide holes 136b of the guide tube 136.

Thereby, the pulling wires 160 are moved forwards along a straight path in the third passages 136a of the guide tube 136, without twisting due to the first and second guide holes 134 and 135 of the guide member 180.

Further, the knob wings 151 are guided to be moved forwards by the third guide holes 136b of the guide tube 136, and thus, the pulling wires 160 are moved forwards along a straighter path in the third passages 136a of the guide tube 136.

Of course, as shown in FIG. 6, the pulling wires 160 may be loosened from the winding portion 121 by automatically rotating the handle 120 in the reverse direction through driving of the driving member 180, without using the user's fingers.

Alternatively, due to release of driving of the driving member 180 through the manual switching button 181, the handle 120 may be rotated manually in the reverse direction.

Thereby, the outer tube 150 may compress the new stent 1 mounted in the mounting space 142.

Thereafter, the knob head 191 is gripped and thereby the iron core 190 is easily inserted into the inner tube 140 and the guide tip 141.

That is, when the iron core 190 is inserted, the inner tube 140 and the outer tube 150 are in a stationary state in which they are not bendable until another operation is performed.

As shown FIGS. 11 to 18, in the stent delivery device 200 according to the second embodiment of the present invention, in order to perform an operation, the knob head 291 is gripped and thereby the iron core 290 is easily removed from the inner tube 230 and the guide tip 231.

When the iron core 290 is removed, the inner tube 230 and the outer tube 240 may be in a free state in which they are bendable corresponding to a lumen in the body.

Thereafter, the guide wire 260 is easily inserted into the inner tube 230 through the extension portion 233, and is exposed from the guide tip 231.

Here, the guide wire 260 is guided by the guide plane 233a of the extension portion 233, thereby being more easily inserted into the inner tube 230.

Thereafter, after a site to be operated on is confirmed by inserting an endoscope into an incision partially formed in the lumen of the body, the guide wire 260 is pushed into the site to be operated on, i.e., a lesion part.

Thereafter, the outer tube 240 is moved to the site to be operated on along the guide wire 260 by pushing the knob 210 and the knob wings 241.

Here, the guide tip 231 connected to the inner tube 230 is caught on the outer tube 240 moved by the knob wings 241, and is thus moved to the site to be operated on along the guide wire 260.

Thereafter, in the state in which a user grips the knob 210, the user rotates the handle 220 and thus slowly pulls the pulling wires 250.

Here, the user's fingers rotating the handle 320 are prevented from slipping on the handle 320 by the knurled parts 222.

Then, the pulling wires 250 are wound on the winding portion 221, thus pulling the connection tip 240a of the outer tube 240.

Here, the outer tube 240 is slowly moved backwards the handle 220 which slowly winds the pulling wires 250 on the winding portion 221.

Further, the pulling wires 250 are moved backwards along a straight path in the second passage 271 of the guide member 270, without twisting due to the first guide holes 213 of the grip 210 and the second guide holes 272 of the guide member 270.

Further, the knob wings 241 are guided to be moved backwards by the third guide holes 273 of the guide member 270, and thus, the pulling wires 250 are moved backwards along a straighter path in the second passage 271 of the guide member 270.

Of course, as shown in FIG. 14, the pulling wires 250 may be pulled by automatically rotating the handle 220 through driving of the driving member 280, without using the user's fingers.

Alternatively, after driving of the driving member 280 is stopped through the manual switching button 281, the pulling wires 25 may be pulled manually rotating the handle 220.

Thereby, the stent 1 mounted in the mounting space 142 is expanded by backward movement of the outer tube 240 achieved winding the pulling wires 250 on the winding portion 221.

That is, the stent 1 is located in the constricted or closed lesion part and is then expanded, by the stent delivery device 200 according to the second embodiment of the present invention.

Thereafter, when installation of the stent 1 is completed, the inner and outer tubes 230 and 240 and the guide wire 260 of the stent delivery device 200 are removed from the lumen in the body, and then a new stent 1 is mounted in the mounting space 232 of the inner tube 230.

Then, the guide wire 260 is pulled out from the inner tube 230, and the pulling wires 250 wound on the winding portion 221 are loosened by rotating the handle 220 in the reverse direction.

Thereafter, the knob wings 241 are gripped and then moved forwards along the third guide holes 273 of the guide member 270.

Thereby, the pulling wires 250 are moved forwards along a straight path in the second passage 271 of the guide member 270, without twisting due to the first guide hole 213 of the knob 210 and the second guide holes 272 of the guide member 270.

Further, the knob wings 241 are guided to be moved forwards by the third guide holes 273 of the guide member 270, and thus, the pulling wires 250 are moved forwards along a straighter path in the second passage 271 of the guide member 270.

Of course, as shown in FIG. 14, the pulling wires 250 may be loosened from the winding portion 221 by automatically rotating the handle 220 in the reverse direction through driving of the driving member 280, without using the user's fingers.

Alternatively, due to release of driving of the driving member 280 through the manual switching button 281, the handle 220 may be rotated manually in the reverse direction.

Thereby, the outer tube 240 may compress the new stent 1 mounted in the mounting space 232.

Thereafter, the knob head 291 is gripped and thus the iron core 290 is easily inserted into the inner tube 230 and the guide 231.

That is, when the iron core 290 is inserted, the inner tube 230 and the outer tube 240 are in a stationary state in which they are not bendable until another operation is performed.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

* Description of Reference Numerals and Marks*

| | |
|---|---|
| 100: stent delivery device | 110: knob |
| 111: first passage | 112: fitting portion |
| 112a: insertion groove | 120: handle |
| 121: winding portion | 122: knurled part |
| 130: guide member | 131: second passage |
| 132: fitting hole | 132a: insertion protrusion |
| 133: insertion hole | 134: first guide hole |
| 135: second guide hole | 136: guide tube |
| 136a: third passage | 136b: third guide hole |
| 140: inner tube | 141: guide tip |
| 142: mounting space | 143: extension portion |
| 143a: guide plane | 150: outer tube |
| 150a: connecting tip | 151: knob wing |
| 160: pulling wire | 170: guide wire |
| 180: driving member | 181: manual switching button |
| 190: iron core | 191: knob head |
| 200: stent delivery device | 210: knob |
| 211: first passage | 212: installation hole |
| 213: first guide hole | 220: handle |
| 221: winding portion | 222: knurled part |
| 230: inner tube | 231: guide tip |
| 232: mounting space | 233: extension portion |
| 233a: guide plane | 240: outer tube |
| 240a: connecting tip | 241: knob wing |
| 250: pulling wire | 260: guide wire |
| 270: guide member | 271: second passage |
| 272: second guide hole | 273: third guide hole |
| 280: driving member | 281: manual switching button |
| 290: iron core | 291: knob head |

1. A stent delivery device comprising:
a knob configured to have a first passage formed therein and a fitting portion formed to protrude therefrom;
a handle rotatably installed on the fitting portion and configured to have a winding portion formed to protrude therefrom;
a guide member configured to have a second passage therein and connected to the first passage, a fitting hole formed on a periphery of the second passage such that the fitting portion is fitted to the fitting hole, an insertion hole formed on the periphery of the second passage such that the winding portion is inserted into the insertion hole, a first guide hole formed through one side of an outer surface of the guide member, and a second guide hole formed through a remaining side of the outer surface of the guide member so as to be connected to the insertion hole;
an inner tube inserted into the first and second passages and then exposed therefrom, and configured to have a guide tip installed on one side thereof so as to form a mounting space in which a stent is mounted;
an outer tube movably installed on the outer surface of the inner tube and configured to have a connecting tip provided on the outer surface thereof; and
a pulling wire connected to the winding portion and the connecting tip of the outer tube through the first and second guide holes.

2. The stent delivery device according to claim 1, further comprising a guide wire configured to be inserted into the inner tube and the guide tip and then to be exposed therefrom.

3. The stent delivery device according to claim 1, wherein a guide tube configured to have a third passage formed therein and connected to the second passage and the first guide hole protrudes from the guide member, and the pulling wire is inserted into the third passage.

4. The stent delivery device according to claim 3, wherein:
a knob wing is installed on the outer tube; and
a third guide hole configured to guide the knob wing is formed through an outer surface of the guide tube.

5. The stent delivery device according to claim 1, wherein:
an insertion groove is formed in an outer surface of the fitting portion; and
an insertion protrusion configured to be inserted into the insertion groove protrudes from an inner surface of the fitting hole.

6. The stent delivery device according to claim 1, wherein the handle has knurled parts formed on an outer surface thereof.

7. The stent delivery device according to claim 2, wherein an extension portion configured to have a diameter greater than a diameter of the inner tube protrudes from one side of the inner tube, into which the guide wire is inserted.

8. The stent delivery device according to claim 7, wherein a guide plane configured to guide the guide wire to the inner tube is formed on an inner surface of the extension portion.

9. The stent delivery device according to claim 1, further comprising a driving member installed in the knob so as to rotate the handle in forward and reverse directions.

10. The stent delivery device according to claim 9, further comprising a manual switching button installed on the knob, and configured to stop driving of the driving member so as to manually rotate the handle.

11. The stent delivery device according to claim 1, further comprising an iron core inserted into the inner tube through the guide tip.

12. The stent delivery device according to claim 11, wherein the iron core has a knob head exposed from the guide tip.

13. A stent delivery device comprising:
a knob configured to have a first passage formed therein and an installation hole and a first guide hole connected to the installation hole, the installation hole and the first guide hole being formed in an outer surface thereof;
a handle configured to have a winding portion rotatably installed in the installation hole so as to protrude therefrom, and exposed from the installation hole;
an inner tube inserted into the first passage and then exposed therefrom, and configured to have a guide tip installed on one side thereof so as to form a mounting space in which a stent 1 is mounted;
an outer tube movably installed on an outer surface of the inner tube and configured to have a connecting tip provided on an outer surface thereof; and
a pulling wire connected to the winding portion and the connecting tip of the outer tube through the first guide hole.

14. The stent delivery device according to claim 13, further comprising a guide wire configured to be inserted into the inner tube and the guide tip and then to be exposed therefrom.

15. The stent delivery device according to claim 13, further comprising a guide member installed on the knob and configured to have a second passage formed therein and connected to the first passage and a second guide hole formed in an outer surface thereof and connected to the first guide hole and the second passage,
wherein the pulling wire is inserted into the second passage and the second guide hole.

16. The stent delivery device according to claim 15, wherein:
a knob wing is installed on the outer tube; and
a third guide hole configured to guide the knob wing is formed through an outer surface of the guide member.

17. The stent delivery device according to claim 13, wherein the handle has knurled parts formed on an outer surface thereof.

18. The stent delivery device according to claim 14, wherein an extension portion configured to have a diameter greater than a diameter of the inner tube protrudes from one side of the inner tube, into which the guide wire is inserted.

19. The stent delivery device according to claim 18, wherein a guide plane configured to guide the guide wire to the inner tube is formed on an inner surface of the extension portion.

20. The stent delivery device according to claim 13, further comprising a driving member installed in the knob so as to rotate the handle in forward and reverse directions.

21. The stent delivery device according to claim 20, further comprising a manual switching button installed on the knob, and configured to stop driving of the driving member so as to manually rotate the handle.

22. The stent delivery device according to claim 13, further comprising an iron core inserted into the inner tube through the guide tip.

23. The stent delivery device according to claim 22, wherein the iron core as a knob head exposed from the guide tip.
